(19) United States
(12) Patent Application Publication
(10) Pub. No.: US 2026/0007475 A1
(43) Pub. Date: Jan. 8, 2026

(54) APPARATUS AND METHOD FOR INTERACTIVE THREE-DIMENSIONAL SURGICAL GUIDANCE

(71) Applicant: Kaliber Labs Inc., San Francisco, CA (US)

(72) Inventors: Aneesh JONELAGADDA, San Francisco, CA (US); Salvatore PENACHIO, San Francisco, CA (US); Chandra JONELAGADDA, San Francisco, CA (US)

(21) Appl. No.: 19/127,410

(22) PCT Filed: Nov. 6, 2023
(86) PCT No.: PCT/US2023/078818

§ 371 Date: May 5, 2025

Related U.S. Application Data
(60) Provisional application No. 63/422,897, filed on Nov. 4, 2022.

(51) Int. Cl. A61B 34/20; A61B 1/00; A61B 90/00; G06T 19/00

(52) U.S. Cl. CPC A61B 34/20; A61B 1/000096; A61B 1/00045; G06T 19/003; A61B 2034/2065; A61B 2090/367; G06T 2210/41; G06T 2219/004

(57) ABSTRACT

An apparatus and method are disclosed for providing interactive 3D surgical guidance that includes a reference 3D map and a live 3D map. Reference 3D maps are navigable anatomy models that enable a surgeon to visualize endoscopic surgical operations prior to actually performing the surgery. Reference 3D maps may be generated from video images collected from prior surgeries. While a surgery is ongoing, a live 3D map may be generated from video images supplied from a surgical camera placed within the patient.

### CLAIM OF PRIORITY

This patent application claims priority to U.S. provisional patent application No. 63/422,897, titled “APPARATUS AND METHOD FOR INTERACTIVE THREE-DIMENSIONAL SURGICAL GUIDANCE,” filed on Nov. 4, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present embodiments relate generally to surgery and more specifically to providing a method and/or apparatus for interactive three-dimensional (3D) surgical guidance.

### BACKGROUND

Minimally Invasive Surgeries (“MIS”) are associated with reduced complications and shorter healing times. However, MIS is cognitively demanding on the surgeon. The surgeon does not see the surgical site, rather the surgeon makes small incisions, operates a scope and surgical tools, and sees the surgical site on a large monitor. This indirection and the lack of binocular sighting of the surgical site forces the surgeon to continuously map the field of view into 3D and perform the procedure. Over time some surgeons develop the ability to create a mental 3D map of the surgical space and gain proficiency in performing surgeries.

However, for most surgeons, this ability takes longer to develop for various reasons, resulting in longer surgery times. Extended time under anesthesia is associated with negative outcomes like postoperative nausea, thromboembolism, postoperative infection, postoperative core hypothermia, cardiopulmonary complications.

In some surgeries, the patients' preoperative diagnostic radiological imaging is used to plan surgeries. The planning could include deciding resection margins, estimating bone losses, etc. However, when these preoperative images are not available in the surgical field of view. Developing a mental map between the preoperative imaging and the surgical field of view is challenging, this skill is only developed over time.

Thus, it would be helpful to provide methods and apparatuses for to provide 3D navigational guidance to surgeons when performing MIS procedures

### SUMMARY OF THE DISCLOSURE

Described herein are systems and methods for providing surgical guidance to a surgeon regarding any feasible surgical procedure. The surgical guidance may include one or more navigable three-dimensional (3D) maps. In some examples, the 3D map may be a reference 3D map that is constructed from a large number of surgical videos that are from patients that have undergone similar procedures. In some other examples, the reference 3D map may be constructed from surgical videos performed on cadavers. Using the reference 3D map, a surgeon can study typical anatomies to prepare him or her for performing any feasible surgical procedure.

In some other examples, the 3D map may be a live 3D map that may be constructed from a surgical video (images) provided by an endoscopic camera inserted and progressing through a patient.

In general, the 3D maps may be generated by a neural renderer working in conjunction with one or more other neural processing networks. The neural renderer may generate the 3D maps from video images while the further neural networks can identify anatomies, pathologies, and/or surgical tools within the 3D maps.

Any of the methods and apparatuses (e.g., systems, including software) described herein may be used to provide surgical guidance with constructed 3D anatomical maps. Any of the methods may include receiving a surgical video of a surgical procedure being performed on a patient, retrieving, based on the surgical video, a 3D map, wherein the 3D map is reference 3D map based on surgical videos of previous surgical procedures, and displaying the 3D map.

Any of the methods described herein may also include generating a live 3D map, based on the surgical video, and displaying an updated 3D map based on the live 3D map

In any of the methods described herein, the updated 3D map may be a blend of the reference 3D map and the live 3D map. Furthermore, in some variations, the updated 3D map may include portions of the live 3D map to replace corresponding objects in the reference 3D map.

In any of the methods described herein, the surgical guidance may include determining a measurement between points on an anatomy surface of the live 3D map. Any of the methods described herein may also include recognizing a pathology in the live 3D map, wherein the recognizing includes executing a neural network trained to recognize patient pathologies. Furthermore, in any of the methods described herein, the live 3D map may be generated in response to recognizing that new regions of a surgical site have been sighted.

In any of the methods described herein, retrieving the 3D map may include executing a neural network trained to recognize anatomy in the surgical video, and retrieving the 3D map based on the recognized anatomy. In any of the methods described herein, the reference 3D map may be a composite of a plurality of 3D maps having at least one common anatomical characteristic.

Any of the systems described herein may include one or more processors and a memory configured to store instructions that, when executed by the one or more processors, cause the system to receive a surgical video of a surgical procedure being performed on a patient, retrieve, based on the surgical video, a 3D map, wherein the 3D map is reference 3D map based on surgical videos of previous surgical procedures, and display the 3D map.

For example, described herein are methods comprising: constructing a three-dimensional (3D) map of a surgical site from one or more scans of the patient; automatically setting a plurality of keypoints based on the shape and/or location of anatomical structures prior to performing the surgical procedure; automatically detecting, during the surgical procedure, at least a subset of the plurality of keypoints in the field of view of a camera inserted into a patient's body; coordinating the 3D map with the field of view using the keypoints; and using the coordinated 3D map to estimate a position and orientation of a surgical tool and/or a camera within the body.

Also described herein are apparatuses and/or software for performing any of these methods. For example, described herein are systems (e.g., systems for providing surgical guidance during a surgical procedure) that may include: one or more processors; and a memory coupled to the one or more processors, the memory storing computer-program instructions, that, when executed by the one or more processors, perform a computer-implemented method comprising: constructing a three-dimensional (3D) map of a surgical site from one or more scans of the patient; automatically setting a plurality of keypoints based on the shape and/or location of anatomical structures prior to performing the surgical procedure; automatically detecting, during the surgical procedure, at least a subset of the plurality of keypoints in the field of view of a camera inserted into a patient's body; coordinating the 3D map with the field of view using the keypoints; and using the coordinated 3D map to estimate a position and orientation of a surgical tool and/or a camera within the body.

Any of these methods and apparatuses may include identifying anatomical structures within the 3D map using an Artificial Intelligence (AI) agent prior to performing the surgical procedure. In some examples constructing the 3D map of a surgical site comprises constructing the 3D map from one or more MRI scans of the patient. Automatically setting the plurality of keypoints may comprise automatically selecting the plurality of keypoints from a library of predetermined keypoints. Any of these methods and apparatuses may include confirming that the 3D map is appropriate for the surgical procedure. Any of these methods and apparatuses may include using the coordinated 3D map to identify, track and/or measure one or more landmarks. Any of these methods and apparatuses may include using the coordinated 3D map to navigate one or more surgical tools within the body.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a schematic block diagram showing an example three-dimensional (3D) surgical guidance system.

FIG. 2 is a flowchart showing an example method for generating a 3D map.

FIG. 3 is a flowchart showing an example method for generating a reference 3D map.

FIG. 4 is a flowchart showing an example method for providing surgical assistance with one or more 3D maps.

FIGS. 5A-5B show example views of a 3D map.

FIG. 6 shows a block diagram of a device that may be one example of any device, system, or apparatus that may provide any of the functionality described herein.

FIG. 7 schematically illustrates one example of a method of preparing a 3D map prior to performing the surgery.

FIG. 8 schematically illustrates an example of a method of using the 3D map as

described herein.

FIGS. 9A-9D illustrate an example of one method of determining camera pose and of detecting landmarks using a 3D map as described herein.

FIG. 10 illustrates an example of a method of identifying landmarks as described herein using a 3D map.

### DETAILED DESCRIPTION

Described herein are systems and methods for providing surgical guidance to a surgeon performing a surgical procedure. The surgical guidance may include a navigable three-dimensional (3D) map. In some examples, the 3D map may be a reference 3D map that is constructed from a large number of surgical videos that are from patients that have undergone similar procedures. In some other examples, the reference 3D map may be constructed from surgical videos performed on cadavers. In some examples the 3D map may be derived from one or more scans (e.g., magnetic resonance imaging, or MRI scans) taken of the patient. Using the reference 3D map, a surgeon can study typical anatomies to prepare him or her for performing any feasible surgical procedure.

In some examples, the 3D map may be a “live” 3D map constructed from (or modified by) video provided by one or more cameras, such as an endoscopic camera currently inserted and progressing through a patient. The live 3D map may provide the surgeon a 3D representation or model to refer to or interact with to enhance treatment provided to the patient.

The 3D map may be determined or constructed remotely (away from) the operating room, in some cases with a cloud-based (network accessible) processor, processing device, processing node, or the like. Usage of the 3D maps may not be limited to the operating room, but may be shared with and used by a variety of users, equipment providers, or other colleagues, for example. Generation of the 3D maps may include the execution of one or more neural networks to identify patient anatomies, pathologies, and surgical tools to assist the surgeon. The 3D map may be initially formed ahead of the surgical procedure, and used and/or modified during the surgical procedure.

In any of these methods and apparatuses (e.g., systems, devices, etc., which may include software, hardware and/or firmware), the initial 3D map may be formed based on one or more anatomical, and in some cases volumetric, scans of the patient's anatomy, such as, but not limited to MRI scans, CT scans, etc. Pre-surgical scans may be pre-processed to include predetermined marks, such as points, regions etc., in some cases referred to as “keypoints,” that may be automatically, manually or semi-automatically (e.g., manually confirmed, suggested, etc.) placed in the 3D map prior to performing the surgery. The keypoints may be based on the shape and/or locations generic across a population of patients. For example, key points may be held in a library or database of common keypoints, and some or all of these may be identified int the 3D map. Examples of keypoints may include commonly recognizable (by one of skill in the art) anatomical positions, such as the apex of the arch of the femoral condyle, an anterior-most or posterior-most position of an organ or internal structure, etc. As described herein, these keypoints may be used to coordinate the initial (pre-surgical) 3D map with the 2D images (e.g., video and/or camera images) taken during the surgical procedure. A data structure including the subset of keypoint identified in a particular patient's 3D map pre-surgery may be used during the surgery.

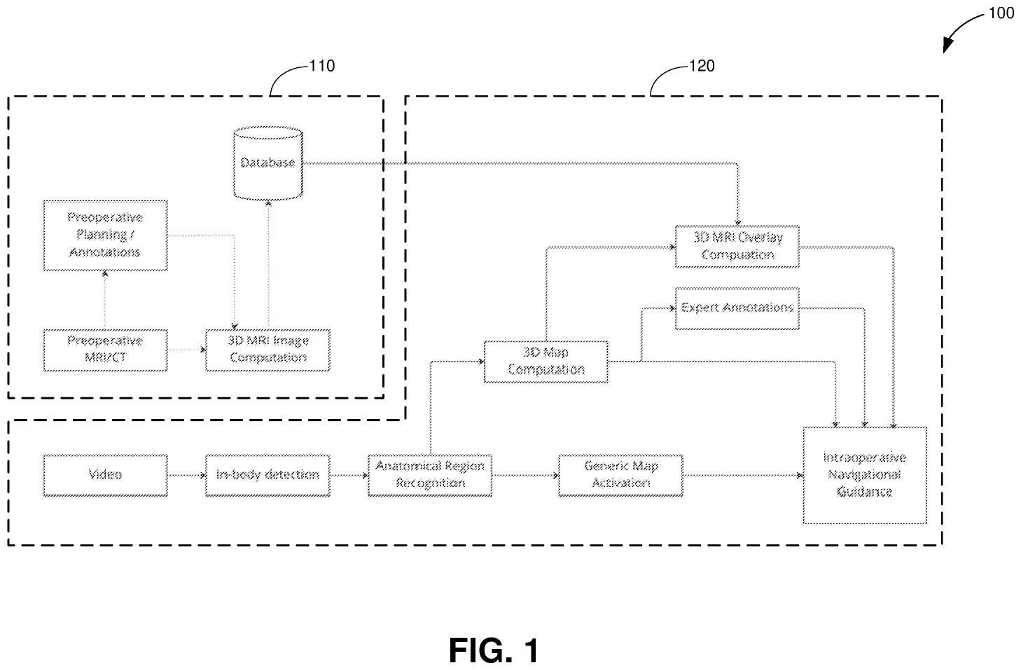

FIG. 1 is a schematic block diagram showing an example three-dimensional (3D) surgical guidance system 100. Although described herein as a system, the 3D surgical guidance system 100 may be realized with any feasible apparatus, e.g., device, system, etc., including hardware, software, and/or firmware. The 3D surgical guidance system 100 is divided into two blocks for the purposes of explanation. Other divisions of the 3D surgical guidance apparatus are contemplated, additional components (modules, engines, database/datastores, etc.) may be included and some of the components shown may be optional.

Block 110 includes blocks representing tasks (and/or associated engines/modules) that are performed or executed before a surgery on a patient. Block 120 includes blocks representing tasks and/or associated modules that are performed during or as part of a patient's surgical procedure. Block 110 may generate a database of one or more reference 3D maps while block 120 uses the generated database (e.g., generated 3D map(s)) as well as incoming video from a live surgery to generate “live” 3D maps. The “live” 3d maps may also be referred to herein as coordinated 3D maps, as the 3D map(s) are coordinated with the 2D images received during the procedure in an ongoing manner, in real time, from the camera images, e.g., video.

In any of these methods an apparatuses, the one or more 3D maps may be coordinated with the 2D images using the keypoints, and in particular, using the subset of key points established during the pre-surgical process 110.

In some examples, block 110 may include a procedure to collect preoperative scans (e.g., images). The preoperative images may include images from a patient's CT scan, MRI, and/or pre-operative endoscopic video. In some variations, the preoperative scans may be annotated by a surgeon or other clinician. In particular, the preoperative scans may be marked to include a plurality of keypoints, and the marked keypoints may be held in a related or associated data structure (such as an index) for use when coordinating with the 2D (e.g., video) images.

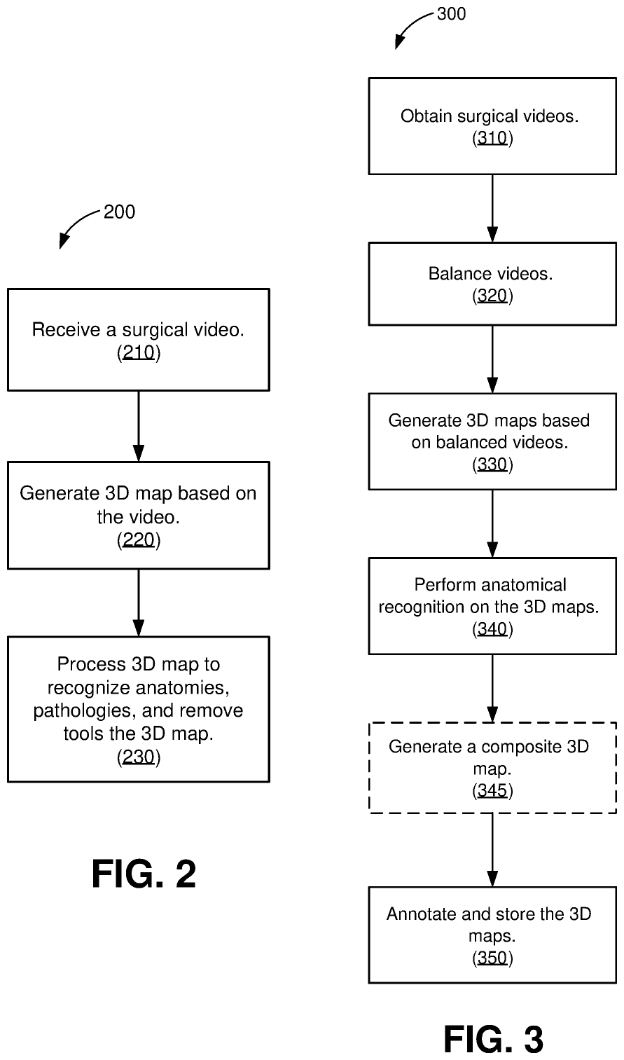

The preoperative images may be provided to a 3D Mapping Image Computation Engine (referred to herein as a 3D Mapper). The 3D mapper can generate a 3D map based on the preoperative images and store them in a database. Operation of the 3D Mapper is described in more detail in conjunction with FIG. 2. The 3D mapper may coordinate the 3D map with the 2D images using the key points.

Block 120 may include modules that receive video, such as endoscopic surgery video. The video may be processed by a module that determines or detects when the images are representative of (or related to) in-body images. In some examples, the video also may be processed by modules that recognize various anatomies within a body. For example, the video may be processed by a machine language module (in some cases, a trained neural network) to recognize different anatomies within the body.

In some examples, after anatomy recognition, a generic 3D map may be activated. The generic 3D map (referred to herein as a reference 3D map) may be based on images that may come from other surgeries and, in some cases, surgical procedures performed on a cadaver. The reference 3D map may be generated in advance of any procedures associated with block 120, and in some cases may be prepared using procedures and methods associated with block 110.

Also after anatomy recognition, a live 3D map is generated (computed) using the received surgical video. The live 3D map may replace or enhance the reference 3D map that is provided as part of an interactive 3D surgical guidance that is provided to a surgeon. In some variations, the live 3D map may be generated with the 3D mapper described with respect to block 110. The live 3D map (e.g., the coordinated 3D map) may be used for navigation, measuring, etc. during and/or after the surgical procedure.

In some examples, a 3D MRI overlay may be computed using the live 3D map and the database generated with respect to block 110. The generated 3D MRI overlay may also be provided as part of an interactive 3D surgical guidance. In some other examples, the live 3D map may receive expert annotations. The annotated live 3D map may then be provided as part of an interactive 3D surgical guidance.

FIG. 2 is a flowchart showing an example method 200 for generating, or in some examples updating, a 3D map, including a coordinated 3D map. Some examples may perform operations of a 3D mapper described herein with additional operations, fewer operations, operations in a different order, operations in parallel, and some operations differently. The method 200, as described herein, may be performed by any suitable apparatus, system, or device.

In some examples, the method 200 may include executing a machine learning and/or deep learning-based algorithm or procedure to generate/coordinate 3D maps from one or more video sources. In some variations, the method 200 may include executing one or more neural networks that have been trained to generate and/or coordinate 3D maps from or using video images (2D images). In general, the 3D map generated/coordinated by the method 200 may determine a camera position as an input and generate a 3D image with respect to that camera position.

The method 200 begins in block 210 as a surgical video is received. For example, the video may be from an endoscopic camera, or any other feasible image capturing device. The surgical videos are from any image-based procedures which may be minimally invasive surgeries. In some other examples, the surgical video may be recorded and retrieved from a memory or other storage device. Next, in block 220 a 3D map may be generated based on the video received or obtained in block 210. In some examples, the video from block 210 may be decomposed into individual frames. The 3D map may be generated/updated with a neural rendering engine (a neural renderer) such as NeRF (Neural Radiance Fields) described by Mildenhall et al. presented at the 16^{th} European Conference of Computer Vision (Mildenhall, B., Srinivasan, P. P., Tancik, M., Barron, J. T., Ramamoorthi, R., & Ng, R. (2020). NeRE: Representing Scenes as Neural Radiance Fields for View Synthesis. ECCV.), although any other feasible 3D rendering engine may be used.

In a NeRF neural network, the input is the camera position, described in terms of 5 degrees-of-freedom in a constrained 3D space, with each dimension corresponding to a different camera orientation. The camera orientation can be described as a 5-dimensional vector composed of coordinates in 3D space and the rotational and azimuthal angles of the camera. This input is fed into a large “multi-layer-perceptron” (MLP), which is a simple neural network consisting solely of fully connected layers, in which each node in a layer is connected to every node in the subsequent layer.

The output of the MLP varies across several neural rendering techniques, but for NeRF it is the red, green, and blue, (RGB), values of a ray and the volumetric density. The difference between NeRF and other neural network-based renderers is that NeRF performs ray tracing based on the volumetric density of the 3D space and RGB output value to form a novel render, i.e., visual representation of the space for a given input camera position. Other techniques simply output a 3D occupancy field of the surface, and rays terminate at the surface.

Thus, for the NeRF output, the image at a given location is determined by numerical integration of several ray tracing functions. It is worth noting that NeRF also maps the input space to a higher frequency space, so that underlying mappings are lower frequency functions, which neural networks tend to learn better.

Additionally, hierarchical sampling is performed which assigns different computational importance to different rays based on the volumetric density-if a ray is just through empty space, for example, it will only be coarsely sampled, but if the volumetric density contains more information, there will be another pass of finer sampling of the ray at around the region of interest. These aforementioned techniques thus serve as optimizations for accuracy and rendering speed.

Execution of NeRF first requires a set of input images with a high number of point correspondences. These point correspondences are matched, and the camera poses (position and orientation of the camera at the time of the initial imaging) are inferred from the COLMAP algorithm (Schönberger, 2016). The COLMAP algorithm outputs a series of camera poses for each input image, and these camera positions combined with the output image is used to train the NeRF neural network. After training the NeRF neural network, new camera poses can be inputted, and output views of the 3D scene can be visualized and rendered (Property 1). These camera poses also exist in 3D, as well as the volumetric density, so one could think of the NeRF algorithm as learning the pointwise 3D structure and optical properties with a granularity determined by the volumetric density (Property 2). This 3D structure's coordinate axes are tied to real space-the camera pose coordinate axes correspond to the volumetric density coordinate system, and the camera pose coordinate system can be tied to real world coordinates and measurements through intrinsic camera parameters. This means that given a particular camera, the intrinsic parameters such as focal lengths of camera in millimeters can be found deterministically and reliably. The learned 3D space of the surfaces and renders are thus tied to real world coordinates regardless of, for example, how close the initial camera is to the surface in the initial images (Property 3). Practically, this means that one can synthesize novel views of a learned structure, as well as understand the underlying 3D character of this structure.

Next, in block 230 the 3D map may be processed to recognize or identify various included anatomies, pathologies, and to remove surgical tools from the 3D map. In some of these examples the 3D map may be coordinated using keypoints that are identified from the 2D images and are present in the pre-surgical 3D map. Thus, the 3D map may be coordinated to the 2D images. In some examples, a processor or processing node may execute a neural network trained to recognize, segment or identity patient anatomy including bones, tendons, joints, musculature, or any other feasible anatomical features in the 3D map. In some variations, the neural networks may be trained with thousands of images of different patient anatomical features. In another example, a processor or processing node may execute a neural network trained to recognize, segment, or identify different patient pathologies included in the 3D map. In yet another example, a processor or processing node may execute a neural network trained to recognize, segment, or identify various surgical tools that may have been captured as part of the received video. After identifying the surgical tools, the surgical tools may then be removed from any subsequent 3D map or image.

FIG. 3 is a flowchart showing an example method 300 for generating a reference 3D map. In some examples, a reference 3D map may be used by surgeons or clinicians to study an operating area prior to the actual procedure. For example, a reference 3D map may be used to rehearse surgical procedures. The rendered view of the reference 3D map may follow a virtual position of a surgical (endoscopic) camera. The reference 3D map may be fully navigable providing a virtual 3D space for the surgeon to explore, practice, and rehearse any surgical procedures.

The method 300 begins in block 310 as one or more surgical videos are obtained. For example a processor or a processing node can access, obtain, or receive one or more surgical videos. In some examples, the surgical videos may be from actual (previous) surgeries that are provided with the patient's consent. In some other examples, the surgical videos may be captured from examination of representative cadaveric samples using similar imaging systems. Cadaveric surgical videos may be used when there is consensus among clinicians that there is substantial equivalence between a surgical video obtained from cadaveric examinations and surgeries on actual patients. Notably, the surgical videos may not be associated with any one particular patient.

Next, in block 320 the videos may be balanced. In some examples, a processor or processing node can execute any feasible computer vision (CV) procedure or method to balance optical characteristics across the surgical videos so that contrast, color, and the like are similar across the various surgical videos obtained in block 310.

Next, in block 330, 3D maps may be generated based on the balanced videos. For example, a processor or processing node can use the balanced videos as input to a 3D mapper such as one described with respect to FIG. 2.

Next, in block 340 anatomical recognition (segmentation) may be performed on the generated and/or coordinated 3D maps. As described above, a processor or processing node may execute a neural network trained to recognize, segment, or identity included patient anatomy including bones, tendons, joints, musculature, or any other feasible anatomical features. In some variations, the neural networks may be trained with thousands of images of patient anatomies. The recognized patient anatomies may be used to describe or label the generated 3D maps. In some variations, the patient anatomy labels may be attached to sections or portions of the 3D map.

Next, in block 345 a composite 3D map may be generated. This step may be optional, as shown with the dashed lines in FIG. 3. Multiple 3D maps may be joined or “stitched” together to form a larger composite 3D map. For example, one or more neural networks may be trained to recognize common anatomies (common anatomical characteristics) in multiple 3D maps and can “stitch” together separate videos based on the common anatomy. In some variations, a composite 3D map may not be larger, but instead include details and characteristics of multiple 3D maps. In this manner, an average or reference 3D map may be constructed.

Next, in block 350 the 3D maps may be stored. For example, the generated reference 3D maps may be stored in a database and may be tagged or labeled with the recognized anatomies. In addition, the 3D maps may also be tagged or labeled with additional descriptors including, but not limited to, access portal information, generic patient/cadaveric information, and the like. The stored 3D maps may be used as reference 3D maps (“gold-standard maps”) that may provide the surgeon with rudimentary or approximate or initial views of a typical patient's anatomy.

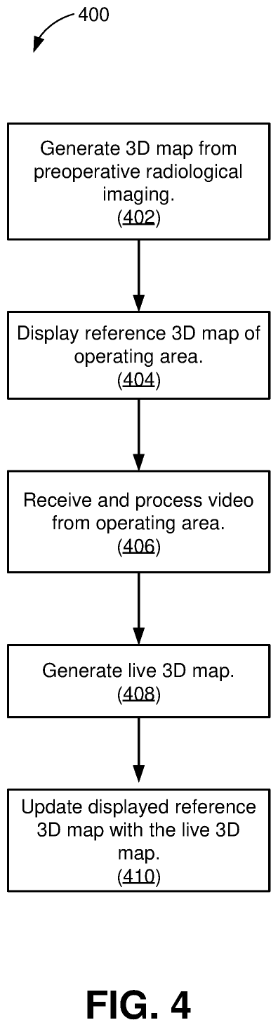

FIG. 4 is a flowchart showing an example method 400 for providing surgical assistance with one or more 3D maps. The method 400 begins in block 402 as a 3D map (e.g., a pre-surgical 3D map or an uncoordinated 3D map) may be generated from preoperative radiological imaging. Preoperative radiological imaging may include any feasible image data including preoperative video from an endoscopic camera, CT, and/or MRI scans. In some embodiments, a 3D map may be generated using a neural renderer as described above with respect to FIG. 2.

Next, in block 404 a reference 3D map of the operating area is displayed. For example, a reference 3D map (a gold-standard map) of the operating area or region may be transmitted and displayed to the surgeon. The reference 3D map may be associated with the anatomy being operated upon by the surgeon and may be generated as described above with respect to FIG. 3. The surgeon may use the reference 3D map to provide initial 3D visualization of the operating area or region. In some cases, the reference 3D map may act as a navigational aid to the surgeon as the surgery begins.

Next, in block 406 video images from the operating area are received and processed. In some examples, a processor or processing node may execute an artificial intelligence program or neural network to determine or detect when an endoscopic camera is actively being used inside a body. The processor or processing node may also execute an artificial intelligence program or neural network to recognize or detect an anatomical region to determine where the surgery and/or procedure is being performed.

Next, in block 408 a “live” 3D map (e.g., coordinated 3D map) may be generated. For example, the video images received in block 406 may be processed according to FIG. 2 to generate a live 3D map of the current operating area. In some variations, the processing of block 406 may trigger the live 3D map generation of block 408. For example, detection of active camera use in block 406 may trigger the live 3D map generation of block 408. Because the live 3D map is coordinated and/or modified based on current up-to-date video images, the live 3D map may provide current 3D visualization to the surgeon thereby assisting in surgical actions and executions. One or more output images may be based on the coordinated 3D map, instead or in addition to the 2D images (video data).

Next, in block 410 the displayed reference 3D map (displayed in block 404) is updated with information from the live 3D map. For example, as the camera enters the body and provides new video images, portions of the reference 3D map may be replaced with information from the live 3D map. In some examples, the live 3D map may replace the reference 3D map on the display entirely, so the surgeon is only presented with current visual data and is not presented with any reference visual data.

In some cases, the reference 3D map may be shaded or tinted differently than the live 3D map enabling the surgeon to discern the difference between actual anatomical structures and reference or “generic” anatomical structures. In this manner, the surgeon is able to discern those anatomical structure that have not yet been sighted on camera. Thus, the display may show an intelligent blend between the reference and live 3D maps.

Updating the 3D map may include determining a position of the camera with respect to an anatomy of the patient and updating the 3D map based on the determined position. In some examples, the NeRF algorithm described in FIG. 2 may be reversed and anatomy recognition may be applied to received video images to determine a position of the camera. Then, the reference 3D map may be rendered and displayed according to the camera position.

In some variations, the patient's radiological images may be projected and/or overlayed onto the reference or live 3D map. In some cases, anatomy recognition algorithms, neural networks, or the like may be applied to the patient's MRI/CT images. In this manner, the correct radiological images are selected and displayed with the appropriate 3D map. In some cases, the surgeon may use this display to determine resection margins or to explore suspicious areas seen in preoperative images. In some cases, the live 3D map may be stored for further study or may be shared with other colleagues, equipment providers, surgeons.

### Extension of the 3D Map for Determining Measurements

In some variations, a 3D map, e.g., a coordinated 3D map, may be used to determine measurements along any anatomy surface. Using tool recognition models, neural networks, and the like described herein, the surgeon can provide input measurement points along a surface in the endoscopic view by positioning a tool in front of the desired points and performing a software-customizable action (such as a key press, foot pedal, etc.). These measurement points can be directly mapped to the surface information learned by the NeRF algorithm (Property 2), and the discrete distance can be computed through numerical integration of the path on the 3D surface through summation of a discretized real-space voxel grid. This distance would be able to account for contours and changes in depth, which adds a higher degree of accuracy and flexibility to the measurement use case.

### Guidance Using the 3D Map

An additional use case of the stitched (composite) 3D map entails surgical navigation of the endoscopic view. Initially, as seen by Property 1 and the COLMAP algorithm: given a view of the surgery, the 3D position of the camera in relation to the 3D scene can be calculated. Then, starting from the stitched representation and the surface anatomy segments of this 3D representation, we use the 3D Scene Graph representation (Armeni et. Al, 2019) to construct a “3D scene graph” of the surgical space.

For example, in an orthopedic shoulder surgery, the 3D map would comprise of nodes, each node corresponding to separate shoulder anatomies such as labrum, glenoid, biceps tendon, etc. These nodes as per the 3D map would have attributes, including but not limited to the 3D mesh and 3D location with respect to a fixed reference frame and coordinate axes, in addition to directional adjacency attributes to other anatomy objects with respect to the same fixed reference frame.

These 3D meshes and 3D locations of the anatomy object are obtained from the stitched representation using anatomy recognition (segmentation) models described herein, providing the gnosis and fixed ontological index of the visible anatomy. The constant-reference frame directional adjacency attributes, i.e., identification of a neighbor along a given direction, can be computed given the constant-reference frame 3D locations. Once these objects/nodes are constructed, the nodes in the 3D map are related through edges. These 3D maps can be queried in real-time as a function of the attributes, much like a geographical map is queries as one drives along a road.

For the case of surgical navigation, one of the desired real-time outputs is the knowledge of where off-screen anatomies are in relation to the current endoscopic view. This is akin to the endoscopic view-specific directional adjacency, which is a function of the 3D scene graph fixed-reference-frame directional adjacency, and the 3D camera position which would then provide a mapping from a 2D endoscopic view to the 3D map. Thus, the scene graph can be queried at any given time given an input view, the 3D camera position can be found utilizing the NeRF procedure combined with anatomy segmentation, and the 3D map (which was constructed using the aforementioned stitched representation of the surgery space through a combination of COLMAP, NeRF, anatomy +pathology segmentation, and tool detection) 3D fixed adjacency can be combined with the 3D camera position to yield a view-specific 2D directional adjacency of anatomies through simple projection back into camera coordinates from 3D coordinates. This adjacency can be visualized as simple arrows with anatomy labels (for example, if at a given view the supraspinatus is to the left offscreen, there will be an arrow on the left side of the screen pointing left with a small label of “supraspinatus”).

An additional use case of the stitched representation (e.g., composite 3D map) and the endoscopic view-3D model correspondence (obtained through either or both of scene graph and Property 2) is merging of stitched representation with regions of interest from MRIs, and navigation cues in the endoscopic view applied to pointing the surgeon towards these locations corresponding to the preoperative MRI region of interested marked a priori. Briefly, the 3D MRI can be matched to the stitched representation, and then using the same navigation principles outlined above, any regions of interest that the surgeons mark or indicated on the MRI before the procedure can be determined in relation to the endoscopic view during the surgery procedure.

Further applications which utilize a combination of the NeRF algorithm pipeline (FIG. 2) with anatomy, pathology, and tool recognition (segmentation) and classification models can be added in the future.

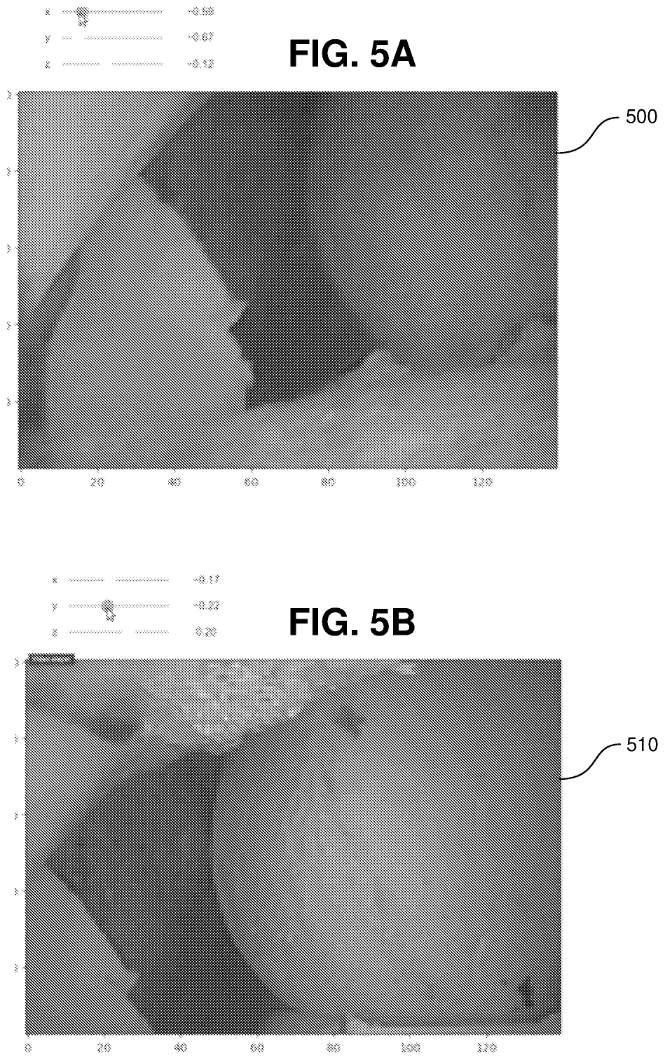

FIGS. 5A-5B show example views of a 3D map. In particular, FIG. 5A shows an example offline interaction with a 3D map 500. FIG. 5B shows a Y coordinate manipulation using an offline 3D map 510.

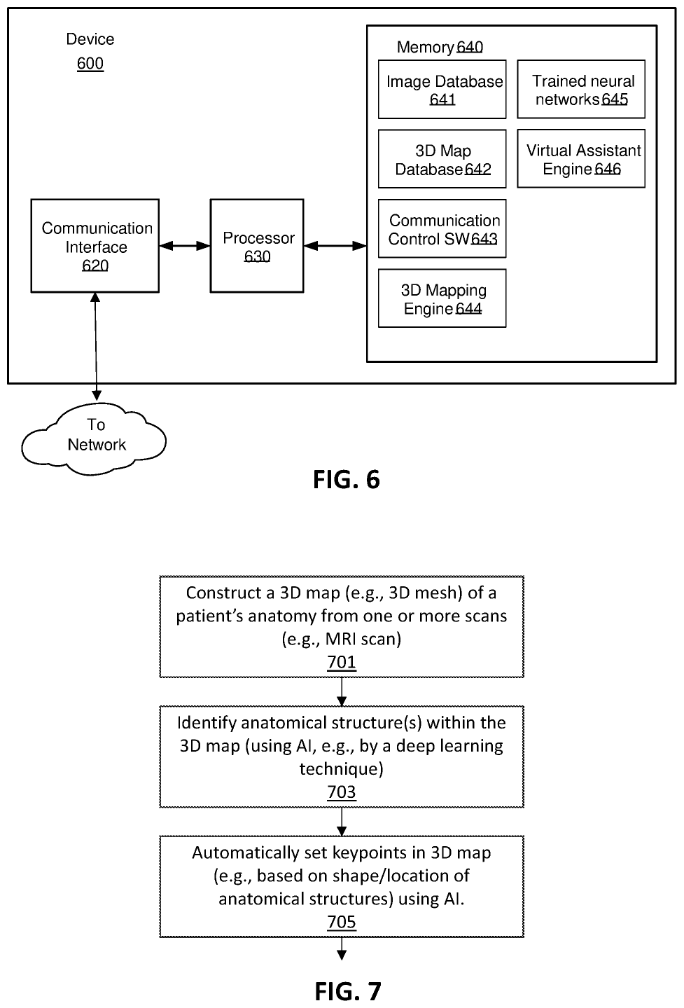

FIG. 6 shows a block diagram of a device 600 that may be one example of any device, system, or apparatus that may provide any of the functionality described herein. The device 600 may include a communication interface 620, a processor 630, and a memory 640.

The communication interface 620, which may be coupled to a network and the processor 630, may transmit signals to and receive signals from other wired or wireless devices, including remote (e.g., cloud-based) storage devices, cameras (including endoscopic cameras), processors, compute nodes, processing nodes, computers, mobile devices (e.g., cellular phones, tablet computers and the like) and/or displays. For example, the communication interface 620 may include wired (e.g., serial, ethernet, or the like) and/or wireless (Bluetooth, Wi-Fi, cellular, or the like) transceivers that may communicate with any other feasible device through any feasible network. In some examples, the communication interface 620 may include surgical video images, CT scans, MRI images, or the like that may be stored in an image database 641 included in the memory 640.

The processor 630, which is also coupled to the memory 640, may be any one or more suitable processors capable of executing scripts or instructions of one or more software programs stored in the device 600 (such as within memory 640).

The memory 640 may include a 3D map database 642 that may include one or more 3D maps. The 3D maps may be any feasible 3D maps including reference 3D maps and live 3D maps as described herein.

The memory 640 may also include a non-transitory computer-readable storage medium (e.g., one or more nonvolatile memory elements, such as EPROM, EEPROM, Flash memory, a hard drive, etc.) that may store the following software modules: communication control software (SW) module 643 to control the transmission and reception of data through the communication interface 620; a 3D mapping engine 644 to generate one or more 3D maps; a trained neural networks module 645 to recognize and/or identify at least anatomies, pathologies, and surgical tools; and a virtual assistant engine 646 to deliver virtual operating assistance to a surgeon. Each software module includes program instructions that, when executed by the processor 630, may cause the device 600 to perform the corresponding function(s). Thus, the non-transitory computer-readable storage medium of memory 640 may include instructions for performing all or a portion of the operations described herein.

The processor 630 may execute the communication control SW module 643 to transmit and/or receive data through the communication interface 620. In some examples, the communication control SW module 643 may include software to control wireless data transceivers that may be configured to transmit and/or receive wireless data. In some cases, the wireless data may include Bluetooth, Wi-Fi, LTE, or any other feasible wireless data. In some other examples, the communication control SW module 643 may include software to control wired data transceivers. For example, execution of the communication control SW module 643 transmit and/or receive data through a wired interface such as, but not limited to, a wired Ethernet interface. Through the communication control SW module 643 and the communication interface 620, the device 600 may provide cloud accessible functionality for any of the operations or procedures described herein.

The processor 630 may execute the 3D mapping engine 644 to generate one or more 3D maps as described above with respect to FIGS. 2-4. For example, execution of the 3D mapping engine 644 may cause the processor 630 to receive surgical video through the communication interface 620 or retrieve surgical video from the image database 641 and generate an associated 3D map. The generated 3D maps may reference 3D maps, live 3D maps, or a combination of reference and live 3D maps. In some examples, the generated 3D maps may be stored in the 3D map database 642.

The processor 630 may execute the trained neural networks module 645 to recognize or identify patient anatomies, patient pathologies, and/or surgical tools. In some variations, execution of the trained neural networks module 645 may enable the processor 630 to recognize or identify when a surgery or surgical procedure is being performed.

The processor 630 may execute the virtual assistant engine 646 to provide surgical assistance to a surgeon. The surgical assistance may include causing the device 600 to receive or obtain one or more surgical videos and generate one or more 3D maps based on the surgical videos using one or more of the modules, interfaces, or the like included within or coupled to the device 600.

### EXAMPLES

A 3D map may be configured as a 3D mesh, e.g., a representation of a solid structure decomposed into polygons, from a patient's MRI and/or other scan. Anatomical structures may be identified in this 3D mesh using, e.g., deep learning techniques on individual slices of sagittal or coronal series, e.g., from the MRI studies in some examples. Other series could be used depending on the location of the structures of interest. During the pre-surgical phase, anatomical structures may be identified from the 3D map, e.g., using subject matter experts and/or computer vision algorithms to delineate and identify structures. For example, a neural networks may be trained to recognize structures and output masks corresponding to various soft and bony structures.

FIG. 7 illustrates an example of a method for preparing a 3D map prior to performing surgical procedure on a patient. In general, a 3D map (e.g., 3D mesh) may be formed of the patient's anatomy 701, as described above. In any of these examples, predetermined points, e.g., keypoints, may be identified on the 3D map (mesh) for a given anatomical region or regions 703. The keypoints may be automatically selected, e.g., by their positions relative to anatomical structures in the 3D map. For example, a keypoint could be at the ‘apex’ of the arch of the femoral condyle at the most anterior portion. In some cases, the keypoints could be at the intersection of two or more anatomical structures at arbitrary points in 3D space, i.e., ‘anterior-most point’, etc. The 3D map may be constructed and the keypoints are located on the 3D mesh prior to the surgery 705.

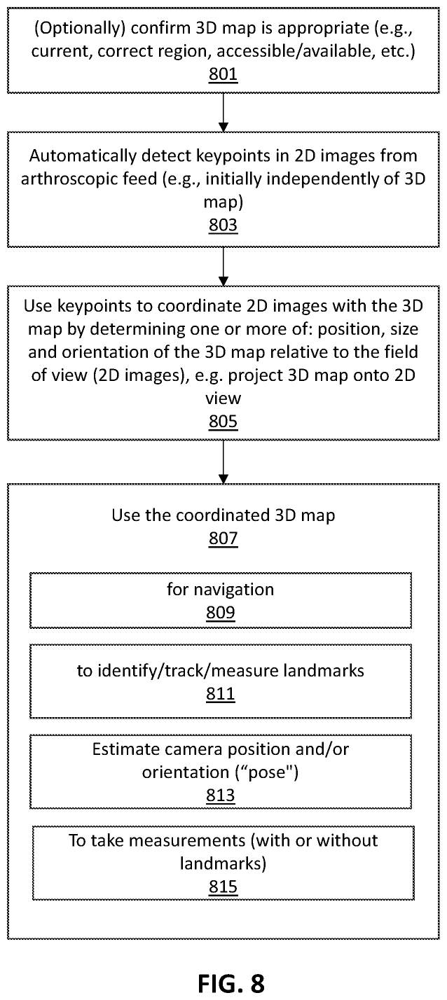

Once prepared, the 3D map may be modified, e.g., using the keypoints, to coordinate with images (2D images, such as, but not limited to, video images) taken during the procedure, as from a scope (endoscope, etc.) to form a ‘live’ or coordinated 3D map. The coordinated 3D map may then be used for assisting in navigation of the surgical devices, measuring one or more regions (including using landmarks) and/or for displaying and otherwise assisting with the surgical procedures. FIG. 8 schematically illustrates an examples of such a method.

At the start of a surgical procedure (or immediately before starting) the method or apparatus may confirm that the pre-surgical 3D map is ready and/or appropriate for use. For example, the timing between the scan(s) used to create the pre-surgical 3D map may be configured to be within a predetermined time period (e.g., less than a few days, weeks, months, year(s), etc.) 801. During the surgery, the arthroscopic feed (e.g., 2D images) may be processed through an anatomical recognition pipeline (e.g., an automated recognition procedure).

In any of these methods an apparatuses a plurality of keypoints, corresponding to the keypoints identified from the pre-surgical 3D map, may be identified in the 2D images 803. This may be done in real time, to locate the keypoints identified a priori in the 3D map, on the images (2D) in the arthroscopic field of view. For example, one or more deep learning techniques such as high-resolution network, HRNET, may be used, e.g., after restricting the region of interest using anatomical structures on which the keypoints are known to reside. Since the keypoints may be chosen in an arbitrary manner on one or more anatomical structures, each keypoint may have an associated set of structured which form the corresponding region of interest.

Once the corresponding keypoints are recognized in the field of view, these methods and apparatuses described herein may use the keypoints in the 2D images to coordinate with the keypoints in the 3D map, e.g., to estimate the position, size, and orientation of the 3D map (from the MRI) in the field of view 805. What the camera perceives may actually be a projection of the 3D mesh onto the 2D field of view. As the 3D map is coordinated with the compound images, the live 3D map (the coordinated 3D map) may be used 807 for a variety of extremely useful aspects to assist in treatment, including, but not limited to: navigation 809, identification/tracking/measuring landmarks 811, estimating camera position and/or orientation (“pose”) 813, to take measurements generally (with or without landmarks) 815, and/or for display. The process of coordinating the 3D map may be ongoing during the medical procedure and/or after the procedure (e.g., post-surgical).

### Landmark detection and tracking

For example, these methods and apparatuses may include pacing and/or detecting (including automatically detecting) one or more landmarks on the 3D map (e.g., MRI images) either pre-surgical (as they are first transferred to the 3D mesh) and/or post-surgical, as corresponding positions in the 2D image are computed since the projection of the 3D mesh has been computed. Landmarks may be placed intraoperatively, i.e., on the 2D field of view, its actual position on the 3D map is computed and its position on the 3D mesh is determined. Once the ‘true’ location of the landmark on the 3D map is determined, the location of the landmark may be tracked even as the field of view changes and the landmark disappears from the field of view.

### Camera Pose Estimation

Any of these methods and apparatuses may use a ‘perspective-n-point (PnP)’ algorithm for estimating the pose of a camera relative to a set of 3D points, given their corresponding 2D image projections. A PnP algorithm may work by solving a system of equations that relate the 3D points and their corresponding 2D image projections. These equations are based on the principles of perspective projection. In general there is just one position of the camera which will produce perfect agreement between 4 or more points between the 2D and 3D views. The PnP algorithms may be solved in real-time, thereby obtaining the position of the camera for each frame of the video.

### Measurements

Measurements may be performed against the 3D mesh. The MRI, with a specified voxel dimension, may establish the scale of 3D map. For example, two or more landmarks may be placed in the field of view, they may be mapped to the 3D space (in the 3D map) from where the distances between them are computed. A reference object of known size is not needed, but may be used for confirmation. For example, the presence of a known object in the field of view may be used to double check the projections and calibrate the 3D mesh.

### Navigation in 3D

The 3D map may include a 3D mesh and/or point clouds of a joint (e.g., knee, shoulder, etc.) or other regions that can be generated using MRI scans. The resulting 3D mesh and/or point clouds may be used as a reference for navigating and also finding the corresponding depth map. Navigation can be solved by estimating the camera position and/or orientation (e.g., pose) in a world coordinate system. The camera pose may include 6 degrees-of-freedom (DOF) which may be made up of the rotation (roll, pitch, and yaw) and 3D translation of the camera with respect to the world. The corresponding depth map of a 2D RGB image may be point clouds as obtained by MRI in the estimated camera pose.

The 3D map (e.g., mesh) may be generated from the scans, including an MRI scan, in any appropriate manner. For example, a 3D map (mesh) may be generated from an MRI scan using inbuilt software, e.g., from 3D slicer/VTK. A general algorithm may be used to generate a 3D mesh from an MRI scan. For example the MRI scan may be segmented. This may involve identifying the different tissues and structures in the MRI. For example, a deep learning model which automatically annotates different anatomical structures may be used. This may result in different anatomical structures that are segmented in the MRI view. In any of these examples, a surface mesh may be generated. Once the different tissues and structures have been segmented, a surface mesh can be generated for each structure. This may be done using a variety of algorithms, such as marching cubes, isosurfacing, or surface reconstruction. The surface mesh may be smoothed and refined. For example, the surface mesh may contain artifacts or irregularities, so it may be smoothed and refined before exporting. This may be done by using a variety of algorithms, such as smoothing filters, decimation, and remeshing.

### Segmentation

Segmentation may include identifying the different tissues and structures in an MRI. For example, a training data set of labeled anatomical structures on the MRI (e.g., labeled manually with sufficient examples) may be provided to train an AI agent (e.g., neural network). Deep learning models like Unet may be used to train the data for semantic segmentation. Once the model is trained, it may be used with the MRIs scans to generate segmentation over a variety of different anatomies and tissues.

Once the different tissues and structures have been segmented, a surface mesh can be generated for each structure. A surface mesh is a 3D mesh that represents the surface of the structure. This may be done, e.g., using a marching cubes technique for generating surface meshes from volumetric data. It works by marching through the volume data and generating a mesh at each voxel where the intensity value is above a certain threshold. Isosurfacing may be used. Isosurfacing is a similar method to marching cubes, but it generates a mesh at all of the voxels where the intensity value is equal to a certain value. Surface reconstruction algorithms may use a variety of techniques to generate surface meshes from point cloud data. These algorithms can be used to generate surface meshes from segmented MRI data, as well as from other sources of point cloud data, such as 3D scans.

The surface mesh forming the 3D map may be smoothed and refined. For example, the surface mesh may contain artifacts or irregularities, so it may be important to smooth and refine the mesh before exporting it. This can be done using a variety of algorithms, such as smoothing filters, decimation, and remeshing. Smoothing filters may be used to remove small artifacts and irregularities from the surface mesh. Decimation algorithms may be used to reduce the number of polygons in the surface mesh without significantly affecting its accuracy. Remeshing algorithms may be used to generate a new surface mesh from the existing surface mesh. This can be used to improve the quality of the mesh or to change the topology of the mesh.

Once the mesh has been smoothed and refined, it may be exported to a variety of formats, such as STL, OBJ, or PLY. A 3D mesh may be obtained from the MRI scan and the 2D RGB image obtained from the scope (e.g., arthroscope) may be used to estimate the camera pose. For example landmark points may be determined. This may be done by using a supervised landmark detector HRNET. In some cases, camera pose may be estimated by using perspective-n-point (PnP) algorithms which uses known 2D and 3D landmarks to establish correspondences. In addition to supervised landmark detector these methods and apparatuses may use deep learning-based Blind PnP algorithms which may not require 2D-3D landmark correspondences.

Any of these methods and apparatuses may refine the camera pose. This may be done, e.g., by Kalman filter and bundle adjustments. For example, these methods may include detecting landmark points. The structures in 2D images as obtained from arthroscopy can be defined by landmark points. These landmark points may be trained specific to the structure which can be aligned with respect to 3D coordinates on the 3D map (e.g., on the 3D mesh). The landmark points in the scene can be trained by supervised learning. The architecture that is being used to train the landmark detection may include a high-resolution network (HRNet), e.g., developed for human pose estimation. It may maintain a high-resolution representations through the whole process by connecting high-to-low resolution convolutions in parallel and may produce strong high-resolution representations by repeatedly conducting fusions across parallel convolutions.

The correspondence between the trained 2D landmark and 3D coordinates on the 3D map may then be established. The 3D coordinates on 3D map (e.g., 3D mesh) may be pre-labeled corresponding to the trained 2D landmarks, so no manual correspondence/registration of the 2D-3D points are required. During the run time, the landmark detection model may predict the landmarks on the 2D which are already registered with 3D coordinates.

Thus, these methods and apparatuses may estimate the camera pose, e.g., using landmarks based PnP techniques. As mentioned, perspective-n-point (PnP) may include a class of algorithms for estimating the pose of a camera relative to a set of 3D points, given their corresponding 2D image projections. PnP algorithms work by solving a system of equations that relate the 3D points and their corresponding 2D image projections. These equations may be based on the principles of perspective projection. Perspective projection is a model of how images are formed in a camera. In perspective projection, the 3D points in the scene are projected onto a 2D image plane using a set of projection rays. The projection rays converge at a single point, called the camera center. The PnP algorithms may use the known 3D points and their corresponding 2D image projections to estimate the camera pose. The camera pose is the camera's position and orientation in 3D space. The camera pose may consist of 6 degrees-of-freedom (DOF) which are made up of the rotation (roll, pitch, and yaw).

These methods and apparatuses may also or alternatively include blind perspective-n-point (PnP) techniques. The blind perspective-n-point (PnP) problem is the task of estimating the pose of a camera relative to a set of 3D points, given their corresponding 2D image projections, without prior knowledge of the 2D-3D correspondences. A deep learning model for solving the blind PnP problem may be used, and may consist of three main components: a feature extractor, a matcher and a pose estimator. The feature extractor may extract features from the 2D image, and the 3D point cloud obtained from the 3d mesh as defined above. A matcher may match the features extracted from the 2D image and the 3D point cloud resulting in matchability matrix. A pose estimator may include feeding the obtained matchability matrix into a classification module to disambiguate inlier matches; once the inlier matches are found, Perspective-n-point (PnP) as described above may be used. To train the deep learning model, a set of training data may be used. The training data may consist of 2D images, 3D point clouds, and the corresponding 2D-3D correspondences.

The estimated camera pose may be refined. For example, the estimated camera pose can be noisy due to several reasons such as noisy frames which may destabilize the supervised and unsupervised landmark algorithms required for establishing correspondences. The refinement may be used to smooth the noises. For example, a Kalman filter and Bundle adjustment may be used to refine estimated camera poses. A Kalman filter (KF) is a recursive algorithm used to estimate the state of a dynamic system from noisy observations. In the context of camera pose estimation, the dynamic system is the camera's motion, and the state is its pose, represented by its position and orientation in 6 degrees of freedom (6 DoF: three for translation and three for rotation). A KF is capable of handling noisy measurements and can provide estimates even when measurements (e.g., PnP readings) are missing for short periods. To use the Kalman filter for camera pose estimation, the method and apparatuses described herein may: define the state vector of the camera pose: the state vector, which includes the camera's position, velocity, orientation, and angular velocity may first be defined. The state transition model may describe how the state evolves over time. For a camera moving in a 3D space, the state transition can be described using kinematics equations. An observation model may come from the PnP outputs, where enough points are observed. The Kalman filter may be initialized with an initial estimate of the camera pose and the covariance of the estimation error. Based on the state transition model, the method or apparatus may predict the next state (6DoF pose) and update the state covariance. The Kalman filter may be updated, e.g., to incorporate the new observations from PnP to correct the predicted pose. This may involve updating the state estimate and updating the state covariance.

In any of these methods an apparatuses the procedure may be repeated, e.g., as part of a loop. For example, the method and/or apparatus may continuously apply the update step as new PnP observations become available. When PnP is not available, simply apply a predicted state based on the kinematics model.

The camera pose may be refined by bundle adjustment. Bundle adjustment is a technique for refining the camera poses and 3D point positions in a set of images. It may be used in computer vision to improve the accuracy of 3D reconstruction. Bundle adjustment works by minimizing the reprojection error. The reprojection error is the difference between the predicted 2D image projections of the 3D points and the actual 2D image points. Bundle adjustment may represent a non-linear optimization problem, which means that it can be difficult to solve.

However, there are a number of efficient algorithms available for solving bundle adjustment problems that may be used. For example, to use bundle adjustment for camera pose and 3D point estimation, the method and/or apparatus may: estimate the initial camera poses and 3D point positions, define the cost function, optimize the cost function and may repeat the steps of defining and optimizing the cost function to minimize. For example, estimating the initial camera poses and 3D point positions may be done using a variety of methods, such as feature matching and PnP. The cost function is a measure of the reprojection error. Optimize the cost function can be done using optimization algorithms. As mentioned these steps may be repeated until the cost function is minimized.

Once the camera pose is established for the specific query frame (e.g., particular surgery or portion of a surgery) as described above, 3D point clouds derived from the 3D mesh obtained from MRI may be used to obtain depth map. For example, these methods and apparatuses may transform 3D Points into Camera Space. To estimate the depth of 3D points in the camera's coordinate system, these methods and apparatuses may transform the 3D points from their world coordinates to the camera's coordinate system using the camera pose. An estimate of depth may be performed by these methods and apparatuses once the 3D points are in camera space. These methods and apparatuses may estimate their depth (Z-coordinate) using the camera's projection matrix and the corresponding 2D image coordinates (u, v) where these points are visible. The camera pose estimation may work by matching known 3D points in a scene to their corresponding 2D image projections, and then using these correspondences to estimate the camera's position and orientation in the 3D space.

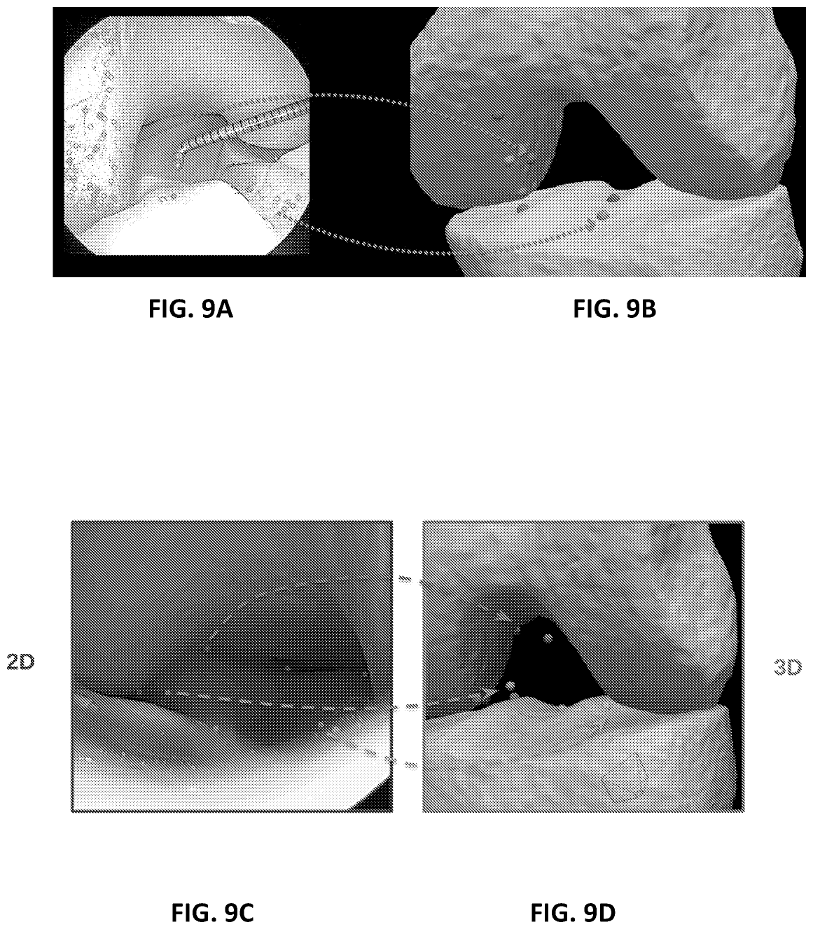

For example, FIGS. 9A-9D illustrate the coordination of a 2D image (FIG. 9A) to a 3D map (FIG. 9B). The dashed arrows in FIGS. 9A-9B illustrate the corresponding points between the two 2D image and the 3D map. Similarly, FIG. 9C is another example of a 2D image that has been manually labeled to identify points on the tibial plateaus across approximately 500 frames of the video. FIG. 9D shows corresponding AP positioned in a 3D view. As in FIGS. 9A-9B, the dashed lines between FIGS. 9C and 9D illustrate corresponding points. Note that the landmarks (points) on the 2D images and corresponding landmarks on the 3D map may be known and fixed a priori. In some cases, the 3D model is assumed to be known, so landmarks can be pre-labelled, as described above. On the 2D image the corresponding landmarks may be inferred based on trained supervised landmark modeling. The camera pose estimation may be achieved by solving PnP between 3D and 2D. There may be at least 4 landmarks in order to solve PnP. In cases where 4 landmarks are not always visible, unsupervised 3D-2D correspondence matching may be used to correct.

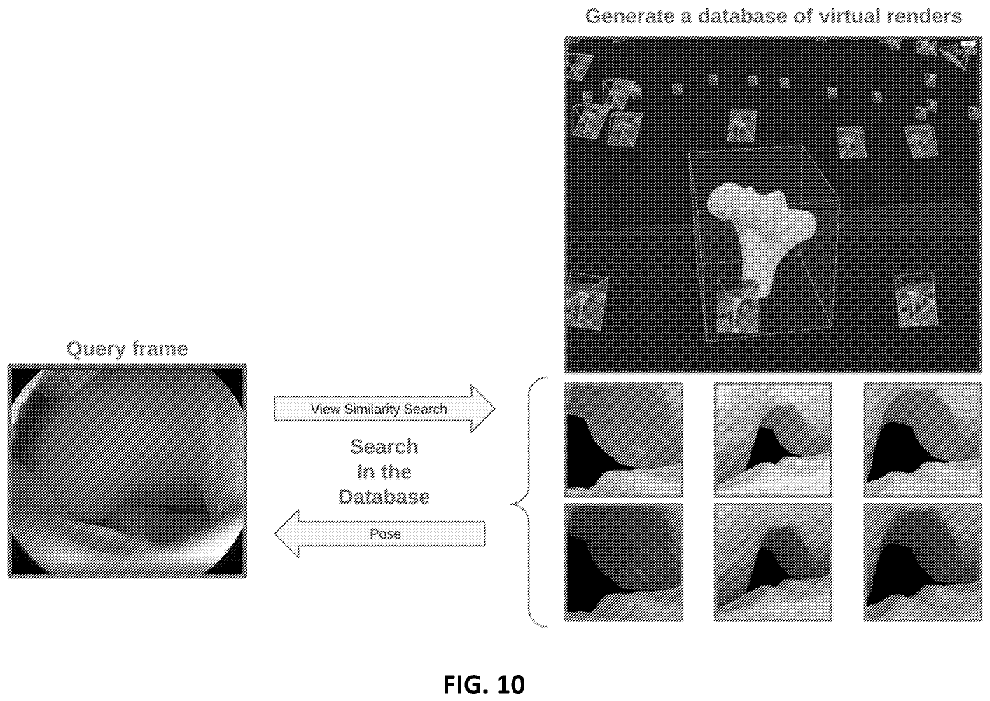

Training supervised landmark models may involve teaching a computer vision model to detect and locate specific landmarks or landmarks in an image or a video frame. Landmarks can represent various objects or features of interest, such as anatomical unique feature points, corners etc. In some examples, HRNet may be used to train landmark detection, as illustrated in FIG. 10. HRNet, short for “High-Resolution Network,” is a deep learning architecture designed for computer vision tasks, particularly semantic segmentation and landmark detection. In this example, unsupervised 3D-2D correspondence may be used to minimize manual AP labeling For example, a query frame (e.g., taken by 2D scan) may be used to search for a similar image in the database, including a database of virtual renders having known poses. Thus, image retrieval may be used in addition to, or instead of, some of the techniques described herein. For example, these techniques may be used to identify the camera pose (position and orientation) as described above.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations, or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of providing surgical guidance during a surgical procedure, the method comprising:
displaying, during the surgical procedure, a three-dimensional (3D) navigation guide of a surgical site to a user, wherein the 3D navigation guide is initially set to a placeholder 3D navigation guide;
taking, by the user, an endoscopic video stream of the surgical site during the surgical procedure;
generating, from the endoscopic video stream of the surgical site, a surgical 3D navigation guide, wherein the surgical 3D navigation guide is navigable and explorable using digital navigational controls;
replacing all or a portion of the 3D navigation guide with the surgical 3D navigation guide so that the user may manipulate and consult the 3D navigation guide during the surgical procedure; and
transmitting a copy of the 3D navigation guide that has been partially or completely replaced with the surgical 3D navigation guide to a party that is remote from the surgical procedure for separate and/or concurrent analysis to form an annotated copy of the 3D navigation guide.

2.-7. (canceled)

8. The method of claim 1, further comprising combining the annotated copy of the 3D navigation guide with the 3D navigation guide.

9.-10. (canceled)

11. A method of providing surgical guidance during a surgical procedure, the method comprising:
displaying, during the surgical procedure, a three-dimensional (3D) navigation guide of a surgical site to a user, wherein the 3D navigation guide is initially set to a placeholder 3D navigation guide;
taking, by the user, an endoscopic video stream of the surgical site during the surgical procedure;
generating, from the endoscopic video stream of the surgical site, a surgical 3D navigation guide, wherein the surgical 3D navigation guide is navigable and explorable using digital navigational controls
wherein generating the surgical 3D navigation guide comprises using Neural Radiance Fields (NRF) to generate the surgical 3D navigation guide; and
replacing all or a portion of the 3D navigation guide with the surgical 3D navigation guide so that the user may manipulate and consult the 3D navigation guide during the surgical procedure

12-15. (canceled)

16. A system for providing surgical guidance during a surgical procedure, the system comprising:
one or more processors;
a memory coupled to the one or more processors, the memory storing computer-program instructions, that, when executed by the one or more processors, perform a computer-implemented method comprising:
displaying, during the surgical procedure, a three-dimensional (3D) navigation guide of a surgical site to a user, wherein the 3D navigation guide is initially set to a placeholder 3D navigation guide;
taking, by the user, an endoscopic video stream of the surgical site during the surgical procedure;
generating, from the endoscopic video stream of the surgical site, a surgical 3D navigation guide, wherein the surgical 3D navigation guide is navigable and explorable using digital navigational controls;
replacing all or a portion of the 3D navigation guide with the surgical 3D navigation guide so that the user may manipulate and consult the 3D navigation guide during the surgical procedure; and
transmitting a copy of the 3D navigation guide that has been partially or completely replaced with the surgical 3D navigation guide to a party that is remote from the surgical procedure for separate and/or concurrent analysis to form an annotated copy of the 3D navigation guide.

17.-22. (canceled)

23. The system of claim 16, further comprising combining the annotated copy of the 3D navigation guide with the 3D navigation guide.

24-25. (canceled)

26. A system for providing surgical guidance during a surgical procedure, the system comprising:
one or more processors;
a memory coupled to the one or more processors, the memory storing computer-program instructions, that, when executed by the one or more processors, perform a computer-implemented method comprising:
displaying, during the surgical procedure, a three-dimensional (3D) navigation guide of a surgical site to a user, wherein the 3D navigation guide is initially set to a placeholder 3D navigation guide;
taking, by the user, an endoscopic video stream of the surgical site during the surgical procedure;
generating, from the endoscopic video stream of the surgical site, a surgical 3D navigation guide, wherein the surgical 3D navigation guide is navigable and explorable using digital navigational controls, wherein generating the surgical 3D navigation guide comprises using Neural Radiance Fields (NRF) to generate the surgical 3D navigation guide;
replacing all or a portion of the 3D navigation guide with the surgical 3D navigation guide so that the user may manipulate and consult the 3D navigation guide during the surgical procedure.

27.-44. (canceled)
